(19) United States
(12) Patent Application Publication
(10) Pub. No.: US 2022/0181004 A1
(43) Pub. Date: Jun. 9, 2022

(54) CUSTOMIZABLE THERAPY SYSTEM AND PROCESS

(71) Applicant: Happify Inc., New York, NY (US)

(72) Inventors: Ran Zilca, Raanana (IL); Tiffany Sun, Brooklyn, NY (US)

(73) Assignee: Happify Inc., New York, NY (US)

(21) Appl. No.: 17/546,020

(22) Filed: Dec. 8, 2021

Related U.S. Application Data
(60) Provisional application No. 63/122,532, filed on Dec. 8, 2020.

(51) Int. Cl. G16H 20/70

(52) U.S. Cl. CPC G16H 20/70

(57) ABSTRACT

The present invention is directed to a computing system and a process carried out by such system for providing a therapy session personalized to the particular circumstances of the user. The therapy session includes an audio component that is automatically generated by an algorithm that makes a voice seem to be that of an actual person. In a similar way, a video component to the therapy session may also be presented.

### FIELD OF INVENTION

The present invention is directed to a computing system and a process carried out by such system for providing a therapy session personalized to the particular circumstances of the user. The therapy session is personalized and particularized including an audio component that is automatically generated by an algorithm that makes a voice seem to be that of an actual person. In a similar way, a video component to the therapy session may also be presented.

### BACKGROUND

This application claims priority to U.S. provisional patent application Ser. No. 63/122,532, filed on Dec. 8, 2020 and incorporated herein in its entirety.

Therapy sessions such as psychotherapy and meditation therapy sessions are often delivered by a human therapist in real-time, i.e., ‘live’. Live therapy sessions of any type are ideal from a number of standpoints but not efficient economically, i.e., they are expensive. Therapy sessions are also amendable to being pre-recorded for delivery at a later time, multiple times or are made available to multiple users to at a time of their choice. While the advantages of pre-recorded therapy sessions, especially from the standpoint of economic efficiency, are readily apparent, there are definite downsides to such pre-recorded sessions. One such downside is the inability to tailor the therapy session to a particular user or users. That is, particular circumstances of a user are often considered by a human therapist delivering therapy in real-time and personalization to the user is very often a significant part of the value provided by real-time therapy sessions.

### OBJECTS AND SUMMARY OF THE INVENTION

In view of the foregoing, it is an object of the present invention to provide a computing system/method for preparing a therapy session and delivering said session in a manner contemporaneous or nearly contemporaneous with its preparation. It is another object of the present invention to provide a computing system/method for achieving a therapy session in the form of a text, audio and video as indistinguishable as possible from a session delivered in real-time by a human therapist.

In accordance with an embodiment of the present invention, a computing system for interacting with a user is provided, in which the computing system commences, with a user, a therapy session. The computing system receives, via at least one sensor, input data relevant to one or both of the user's circumstances and mental state. The computing system generates at least a portion of a therapy session based on the input data relevant to the user. The computing system transforms the portion of the therapy session to be delivered to the user into an audio voice signal that is delivered to the user via an audio output device attached to the computing system such as headphones, earphones, speakers or the like.

An aspect of the present invention is that “circumstances of the user” includes their personality traits, strengths, relationships, support network, life events, varied preferences, physical location, geography and similar information.

An aspect of this embodiment includes ability to generate a real-time voice clone to deliver a therapy session. The term ‘clone’ refers to the goal of having the audio signal that is generated, i.e., the ‘voice’, be as indistinguishable as possible from a human being speaking in real-time. Real-time generation of the audio signal from the clone (model) makes it possible for the therapy to be generated by the computing system to fit as precisely as possible the current circumstances, mental state, etc., of the user.

Another aspect of this embodiment includes the ability to generate a real-time video to accompany the audio voice clone. The video will be synched with the audio signal such that the combination will be as indistinguishable as possible from a video of a human being speaking in real-time. Real-time generation of the video signal from the clone (model) makes it possible for the therapy to be generated by the computing system to fit as precisely as possible the current circumstances, mental state, etc., of the user.

An aspect of the embodiment involves a voice clone model created by taking voice recordings from a therapist. Video recordings synched with the voice recordings may also be captured. Text transcriptions of these voice recordings may be created, e.g., generated by automatic speech recognition, if they do not already exist. The voice recordings, video and text transcripts are passed through an algorithm that uses the voice recordings, video and text transcripts to learn to generate the speech audio and video signal from text. This process is referred to as training. In an embodiment, the algorithm utilized by the computing system also captures the specific personality, tone and other vocal expressive characteristics of the speaker's voice and movements. This is why the process is also referred to as “voice cloning” and “video cloning”.

The therapy session may include behavior intervention.

The therapy session may be a meditation.

As an aspect of this embodiment, the computing system continues to receive, via the one or more sensors, additional input data from the user during the therapy session. The additional input data is continuously utilized by the computing system to generate additional portions of a therapy session. That is, development of additional data regarding the psychological state and circumstances of the user as well as alterations in either during the therapy session is continuously utilized to generate portions of a therapy session.

As another aspect, the therapy session further includes a programmed branching logic for responding to the received input data.

As a further aspect, the behavior intervention is designed to cause an increase in level of happiness of the user.

As a further aspect, the behavior intervention is an activity from a plurality of activities belonging to a Happiness track selected by the user from a plurality of selectable Happiness tracks, wherein each Happiness track is a distinct course of program designed to cause an increase in level of happiness of the user.

As yet another aspect, the behavior intervention is designed to cause a change in one or more of the user's behaviors.

As yet a further aspect, the received input data comprises at least one of verbal and text data from the user.

As still yet another aspect, the semantic analysis includes pre-training a natural language classifier based on a database of user input data and the classifier creating one or more labels to be associated with each of the plurality of conditions.

As a feature of this aspect, the semantic analysis includes determining whether the terms identified in the received input data correspond to the one or more labels.

As a further feature of this aspect, the method further comprises displaying information for viewing by the user.

These and other objects, advantages, aspects and features of the present invention are as described below and/or appreciated and well understood by those of ordinary skill in the art. Although specific advantages have been enumerated above, various embodiments may include some, none, or all of the enumerated advantages and other technical advantages may become readily apparent to one of ordinary skill in the art after review of the following figures and description.

### BRIEF DESCRIPTION OF THE FIGURES

FIG. 1 is a block diagram of an exemplary computing system in accordance with the present invention.

FIG. 2 is an exemplary flow chart showing an overview of steps carried out by an exemplary embodiment of the present invention.

### DETAILED DESCRIPTION

The present invention is directed to a computing system, as well as a method employed by a technological device, that provides an environment for interacting with a human user via various types of therapy. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

The term “therapy” as used herein is intended to be construed broadly to include a variety of therapies that are capable of taking the form of a narrative presentation or a narrative dialogue. Examples of such therapies include one or more of meditation, mindfulness, physical, occupational, relationship, stress, grief, marriage and psychological therapies as well as behavioral interventions.

The term “behavioral intervention,” or just simply, an “intervention” as used herein is intended to be construed broadly, and as such, the term may include a variety of interventions that are designed specifically to increase physical and/or emotional well-being of a user/patient. In accordance with the present invention, an “intervention” may simply be an activity, based on prior evidence-based research, showing that when a person engages with the activity (as intended), the person benefits in terms of his or her psychological and/or physical well-being. In accordance with the present invention, a computing system “provides” a user with an intervention. Generally, this terminology is intended to mean that the computing system loads an intervention, i.e., a stored executable program or a mobile application and commences and/or engages in the user in a set of activities. An intervention is generally comprised of a set of pre-arranged activities or conversations or tasks to be carried out or otherwise performed either by the user or between the user and a coach (or a virtual coach). An intervention also generally has a purpose of activating certain mental or physical mechanisms within the user's mind and/or body, by bringing out certain emotional reactions from the user. As such, an intervention generally comes with an intended implementation, that is, a specific method or approach intended by a creator of such intervention for the set of pre-arranged activities to be carried out in order to most efficiently achieve the underlying purpose behind the intervention. The intended implementation may come in the forms of criteria, conditions, requirements, or factors that are each designed to be met by the user by performing a specific act or speaking a specific word. Accordingly, the most ideal and efficacious way to advance an intervention is for the user to stay faithful to the intended implementation through the course of the intervention.

In accordance with various embodiments of the present invention as described herein, an intervention may be used to train a user to develop certain skills or to modify certain habitual behaviors to address an issue that the user is facing in life. For example, such interventions may include behavioral-change interventions, positive interventions, and clinical interventions (such as Cognitive Behavioral Therapy (CBT), Acceptance and Commitment Therapy (ACT), Solution Focused Therapy (SFT), Behavior Activation (BA), progressive muscle relaxation (PMR), mindfulness based stress reduction (MBSR), and Behavior Change Interventions. Types of meditation that may be delivered include mindfulness meditation, spiritual meditation, focused meditation, movement meditation, mantra meditation, transcendental meditation, progressive relaxation, visualization meditation and loving-kindness meditation. Further in accordance with the present invention, such interventions may be of variable lengths, since the computing system, as will also be described herein, dynamically decides how to continue the interaction at each turn of the intervention based on an assessment of the user's adherence to the intended implementation of the intervention.

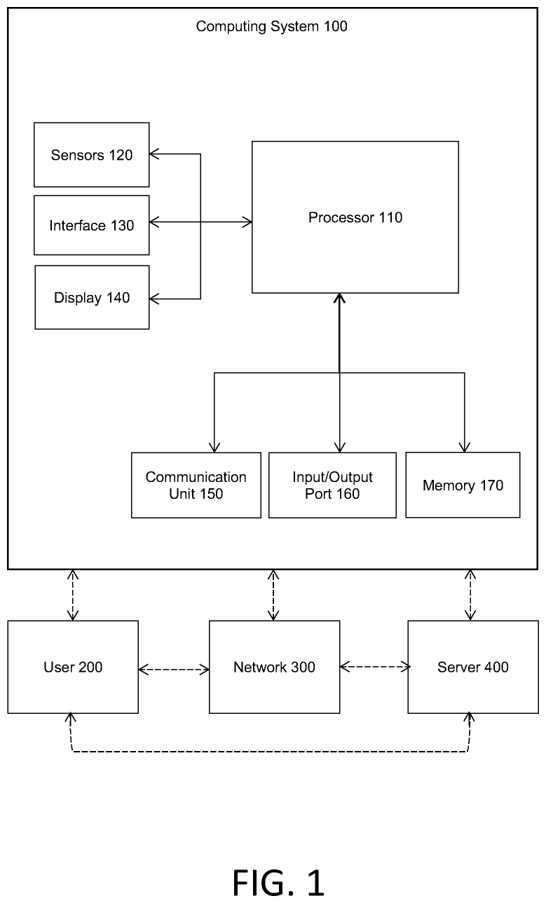

Referring now to the drawings in which like numerals represent the same or similar elements, and initially to FIG. 1 thereof, a computing system 100 configured in accordance with the present invention is illustratively shown in accordance with one embodiment. The computing system 100 includes one or more processors 110 that processes various input data and stored data and controls operations of other components within the computing system 100 to enable the herein described “behavior intervention” between a user or users 200 and the computing system 100. As will be further described, the processor 110 processes data by performing numerous mathematical algorithms and analytical computations. The processor 110 may also be a plurality of processing units that each carries out respective mathematical algorithm and/or analytical computation. In some embodiments, the processor 110 is enhanced by artificial intelligence.

The computing system 100 further includes a plurality of sensors 120. The plurality of sensors 120 may comprise a speaker/microphone, a still image camera, a moving image camera, a biometric sensor, etc. Each of the sensors 120 is configured to obtain user input data and may further comprise one or more respective processing units to process the obtained input data in conjunction with the processor 110. The computing system 100 further includes an interface 130 to allow the user 200 to operate the computing system and a display 140 to present information to the user 200. In some embodiments, the interface 130 and the display 140 may come as one unit such as a touch screen display.

The computing system 100 further includes a communication unit/device 150, an input/output port 160 and a memory 170. The communication unit/device 150 allows the computing system 100 to communicate with the user's other electronic devices or with additional sensors within a vicinity of the user 200 over a network 300. The network 300 may include wireless communications, wired communications, etc. The network 300 may include the Internet, a wide area or local area network, etc. The computing system 100 may use the I/O port 160 for inputting and outputting data. The computing system 100 further includes the memory 170 which stores programs and applications. The memory 170 may store a database of interventions or may locally store interventions retrieved from a server 400 having thereon a database of interventions.

The computing device 100, as well as the user's other electronic devices or the additional sensors, may be part of or otherwise be connected to the network 300 and coupled to a server or a service provider 400. The broken lines in FIG. 1 signify that the user 200, the network 300, the server 400 and the computing system 100 may be connected to any one or more of the user 200,the network 300, the server 400 or the computing system 100, either directly, indirectly, or remotely over a communication path. One or more of the computing system 100, the network 300 and the server 400 may be located on one computer, distributed over multiple computers, or be partly or wholly Internet-based.

In accordance with certain exemplary embodiments of the present invention, the computing system embodies a positive psychology service referred to herein as “Happify.” Happify is a novel, science-based online service for engaging, learning and training the skills of happiness. Happify is based on a framework developed by psychologists and researchers in a collection of therapeutic disciplines such as CBT, Mindfulness, Positive Psychology etc., and assists users in the development of certain skills related to being happy, for example, Savor, Thank, Aspire, Give and Empathize (or STAGE™). In certain embodiments, each skill is developed using various activities, ordered in increasing skill level, that gradually unlock as the user progresses in building that skill. With Happify, a user may select a “track” that contains sets of activities that are designed to address a specific life situation or goal.

The Happify system may be implemented on a user's mobile electronic device, such as asmartphone or tablet, or may be implemented on the user's personal computer (PC). Happify may be embodied within a mobile application, an executable software program, or another suitable form. For instance, a user may download and install a mobile application that provides the Happify service. The user, via the mobile application, selects a Happiness track and is provided with sets of activities that are designed to improve the user's happiness level in accordance with the selected track.

Further details of the Happify system and operations of the Happify system are set forth in U.S. patent application Ser. No. 14/284,229, entitled “SYSTEMS AND METHODS FOR PROVIDING ON-LINE SERVICES,” U.S. patent application Ser. No. 14/990,380, entitled “DYNAMIC INTERACTION SYSTEM AND METHOD,” and U.S. patent application Ser. No.15/974,978, entitled “SYSTEMS AND METHODS FOR DYNAMIC USER INTERACTION FOR IMPROVING HAPPINESS,” and the entire contents of each of these applications is incorporated herein by reference.

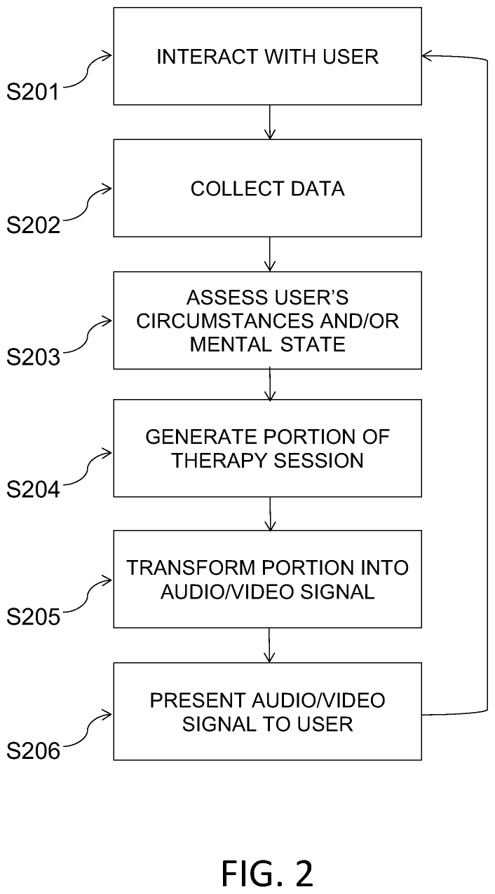

An overview of the steps carried out by an exemplary computing system in accordance with the present invention is shown in FIG. 2. Step S201 entails interacting with a user in an iterative way (i.e., engaging in a conversation either via text or via voice). For example, an iterative interaction initiated by the computing system may comprise providing a user with a prompt, receiving input data from the user, providing a follow-up prompt to the user, receiving further input data from the user, etc. Step S201 need not precede the remaining steps, as will befurther discussed below.

Step S202 entails collecting data from an array of sensors that extract information regarding the user during the interaction. For example, the computing system may be in wired or wireless communication with one or more devices configured to collect user information such as a camera, speaker, microphone, heat sensor, motion sensor, fingerprint detector, keyboard, etc. Such devices may encompass various structures and/or functionalities, and may further include one or more processors to perform various natural language understanding tools.

The data collected by step S202 may include known facts, conditions, psychology, circumstances of the user or users participating in the therapy. The circumstances of the user includes their personality traits, strengths, relationships, support network, life events, varied preferences, physical location, geography and similar information. Input data may be received orderived from one or more sensors associated with the user. GPS, IP address, mobile tower, geofencing, other location data, etc., may reveal the user has not left their house for several days or, conversely, has had several days of non-stop activity, whether potentially indicative of a pathology or merely reflecting a hyperkinetic, not pathological, period. Sleep monitors may also be used as sensors. In addition, mobile devices such as smart phones may function as sleep monitors, being a proxy for wakefulness. That is, actual interaction with the mobile device by the user means that the user is not asleep. Physiological monitors, e.g., smart watch, Fitbit, blood pressure/heart rate monitor, glucose trackers, may also be utilized as sensors. Many physiological monitors are also de facto sleep monitors. Sensors also encompass more traditional inputs like text and voice-to-text input from the user.

The potential exists for S201 and S202 to overlap in a number of areas as well as for one to occur while the other does not occur. Either way, the end product of S201 and/or S202 is the data used as an input to S203. Step S203 entails the assessment of data received from S201 and S202. This data may also be supplemented by data relevant to the user saved in memory 170, e.g., data from previous interactions between the processor 110 and the user. All or some of the data relevant to the user may be used by processor 110 in order to assess the mental state of the user or other circumstances being confronted by the user.

In step S204 the circumstances/mental state of the user is used as an input to the processor. From this information, a therapy narration may be generated by the processor 110. This therapy narration may be sent to the user in the form of text or converted into an audio signal and orally delivered by a person. The narration may also be sent to the user in the form of a combined audio and video signal of a human therapist delivering the text of the therapy.

The conversion of the therapy narration generated at S204 into an audio voice signal and, optionally, a video signal is performed by the processor at S205. At S206, the computing system 100 delivers the audio voice signal to the user, optionally with video. After or during the delivery of the audio/video signal to the user at S206, the process can be sent back to S201 or S202 to gather further information on the user so that additional portions of the therapy session may be generated.

An embodiment of the present invention involves generation of a real-time voice clone as a result of S205. Step S205 may involve utilize a high-level text-to-speech software package. The text-to-speech software package may be trained with a digital database prepared from numerous hours of narration from a human narrator. That is, the voice clone may begin with a collection of digitized oral presentations, e.g., therapy sessions including psychotherapy and meditation sessions. The efficacy of the voice clone may be increased by using as the collection of digitized oral presentations close in tone and format to the voice clone session being prepared. That is, in the example where the therapy session is a meditation, preparing the voice clone from a collection of meditations will increase the quality of the ultimate voice clone product. The video clone may also be generated at S205 and involve an analogous training digital database prepared from hours of video of the human narrator presenting narration.

The AI generated meditation narration may be, as discussed previously, tailored to the known circumstances/mental state of the user(s). This narration may be delivered in real-time, i.e., practically instantaneously. The opportunity for ‘real-time’ generation of the voice signal and video signal from the clone (model) makes it possible for the meditation to be computer generated to fit as precisely as possible the current circumstances, mental state, etc., of the user.

The tailoring of a meditation or other therapy narration may have a number of inputs including known facts, conditions, psychology, circumstances of the user or users participating in the meditation. Further inputs may be derived one or more ‘sensors’ associated with the user. GPS, IP address, mobile tower, geofencing, other location data, etc. That may reveal the user has not left their house for several days or, conversely, has had several days of non-stop activity, whether potentially indicative of a pathology or merely reflecting a hyperkinetic, not pathological, period. Sleep monitors, including mobile device use being a proxy for wakefulness. Many of the physiological monitors below are also de facto sleep monitors. Physiological monitors, e.g., smart watch, Fitbit, blood pressure/heart rate monitor, glucose trackers. Sensors includes more traditional inputs like text and voice-to-text input from the user, whether or not responsive to an Anna based interaction.

Use of ‘voice clone’ technology, with or without an accompanying ‘video clone’ component, has the potential to provide a meditation session that is both personalized and relevant to the particular, day-to-day circumstances of the user is one embodiment of the present invention. For example, if the user is showing signs of depression then the meditation may contain portions designed to help the user deal with this depression. If the person has been exposed to known toxic/triggering person or event(s) then the meditation can proceed through techniques for processing, managing, recovering from and/or otherwise dealing with such things, etc.

The voice clone model may be created by taking voice recordings from a speaker, along with their text transcriptions or have those generated by automatic speech recognition, and passing them through an algorithm that learns to generate the speech audio signal from text. This process may be referred to as “training” the model. The algorithm may capture the specific personality and vocal expressive characteristics of the speaker's voice. This is why the process is also referred to as “voice cloning”. The process for preparing a “video clone” is very similar, with video accompanying the voice recordings through the algorithms, database, etc.

Appearances of the phrase “in an embodiment” or “in an exemplary embodiment,” or any other variations of this phrase, appearing in various places throughout the specification are not necessarily all referring to the same embodiment, and only mean that a particular characteristic, feature, structure, and so forth described in connection with the embodiment described is included in at least one embodiment.

The technology described herein may be incorporated in a system, a method, and/or a computer program product, the product including a non-transitory computer readable storage medium having program instructions that are readable by a computer, causing aspects of one or more embodiments to be carried out by a processor. The program instructions are readable by a computer and can be downloaded to a computing/processing device or devices from a computer readable storage medium or to an external computer or external storage device via a network, which can comprise a local or wide area network, a wireless network, or the Internet.

Additionally, the network may comprise wireless transmission, routers, firewalls, switches, copper transmission cables, optical transmission fibers, edge servers, and/or gateway computers. Within the respective computing/processing device, a network adapter card or network interface in each computing/processing device receives computer readable program instructions from the network and forwards the computer readable program instructions for storage in a computer readable storage medium.

As used herein, a computer readable storage medium is not to be construed as being transitory signals, such as radio waves or other freely propagating electromagnetic waves, electromagnetic waves propagating through a waveguide or other transmission media, or electrical signals transmitted through a wire. The computer readable storage medium may be, but is not limited to, e.g., a magnetic storage device, an electronic storage device, an optical storage device, a semiconductor storage device, an electromagnetic storage device, or any suitable combination of the foregoing, and can be a tangible device that can retain and store instructions for use by an instruction execution device. The following is a list of more specific examples of the computer readable storage medium, but is not exhaustive: punch-cards, raised structures in a groove, or other mechanically encoded device having instructions recorded thereon, an erasable programmable read-only memory, a static random access memory, a portable compact disc read-only memory, a digital versatile disk, a portable computer diskette, a hard disk, a random access memory, a read-only memory, flash memory, a memory stick, a floppy disk, and any suitable combination of the foregoing.

The operations of one or more embodiments described herein may be carried out by program instructions which may be machine instructions, machine dependent instructions, microcode, assembler instructions, instruction-set-architecture instructions, firmware instructions, state-setting data, or either source code or object code written in any combination of one or more programming languages, including an object oriented programming language such as, but not limited to, C++, and other conventional procedural programming languages.

The program instructions, as will be clear to those skilled in the art from the context of the description, may have the capability of being executed entirely on a computer of a user, may also be executed partly on the computer of the user, partly on a remote computer and partly on the computer of the user, entirely on the remote computer or server, or as a stand-alone software package. In the “entirely on the remote computer or server” scenario, the remote computer may be connected to the user's computer through any type of network, including a wide area network or a local area network, or the connection may be made to an external computer. In some embodiments, electronic circuitry including, e.g., field-programmable gate arrays, programmable logic circuitry, or programmable logic arrays may execute the program instructions by utilizing state information of the program instructions to personalize the electronic circuitry, in order to perform aspects of one or more of the embodiments described herein. These program instructions may be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. These program instructions may also be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The computer readable program instructions may also be loaded onto a computer, other programming apparatus, or other device to produce a computer implemented process, such that the instructions which execute on the computer, other programmable apparatus, or other device implement the functions/acts specified in the flowchart and/or block diagram block or blocks.

The block and/or other diagrams and/or flowchart illustrations in the Figures are illustrative of the functionality, architecture, and operation of possible implementations of systems, methods, and computer program products according to the present invention's various embodiments. In this regard, each block in the block and/or other diagrams and/or flowchart illustrations may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently or sometimes in reverse order, depending upon the functionality involved. It will also be noted that each block of the block and/or other diagram and/or flowchart illustration, and combinations of blocks in the block and/or other diagram and/or flowchart illustration, can be implemented by special purpose hardware-based systems that perform the specified functions or acts or carry out combinations of special purpose hardware and computer instructions.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. To aid the Patent Office and any readers of any patent issued on this application in interpreting the claims appended hereto, applicant wish to note that applicant does not intend any of the appended claims or claim elements to invoke 35 U.S.C. 112(±) unless the words “means for” or “step for” are explicitly used in the particular claim.

In view of the foregoing disclosure, an inventive computing system and technique for interacting with users have been described. In accordance with the disclosure provided herein, a computing system engages with users using a behavior intervention, for the purpose of improving levels of happiness, or more broadly, to alleviate or reduce symptoms of mental health conditions such as depression and anxiety, such interaction entailing assessment of adherence fidelity to the behavior intervention by the computing system, to maximize efficiency of the behavior intervention. In further accordance with the disclosure provided herein, the computing system makes assessments of adherence fidelity and dynamically tailors prompts during the behavior intervention to guide the user toward maximized adherence.

What is claimed is:

1. A customizable therapy system, comprising:
a therapy generator operating on one or more servers, the therapy generator automatically generating a therapy session for delivery to the user, the therapy session comprising an audio component and being personalized to a user;
a user computing device selected from the group consisting of a smartphone, tablet, personal computer and smart device, the user computing device receiving input data from the user, the input data supplied to the therapy generator and utilized by the therapy generator to personalize the therapy session;
the user computing device receives the therapy session from the therapy generator and delivers the audio component of the therapy session to the user;
wherein the audio component is an audio voice signal that is delivered to the user nearly contemporaneously with the generation of the therapy session by the therapy generator; and
further wherein the audio component of the therapy session is as indistinguishable as possible from a human delivered therapy session.

2. The customizable therapy system of claim 1, wherein the input data from the user comprising one or more of the circumstances of the user selected from the group consisting of personality traits, strengths, relationships, support network, life events, varied preferences, physical location and geography.

3. The customizable therapy system of claim 1, further comprising:
a. The therapy session also including a video component synched with the audio component, wherein the combination of audio and video is as indistinguishable as possible from a video of a human speaking.

4. The customizable therapy system of claim 1, wherein the audio component is generated from voice recordings.

5. The customizable therapy system of claim 4, wherein generation of the audio component involves the therapy generator generating a therapy text that is passed through an algorithm to convert the text to the audio voice signal utilizing the voice recordings.

6. The customizable therapy system of claim 1, wherein the input data from the user is supplied to the therapy generator during the therapy session.

7. The customizable therapy system of claim 1, wherein the therapy session is selected from the group consisting of meditation therapy, behavioral therapy, yoga therapy, coaching therapy, cognitive behavioral therapy, acceptance/commitment therapy, solution focused therapy, behavior activation therapy, mindfulness based stress reduction therapy and behavior therapy.

8. A computing system for interacting with a user, the system comprising:
one or more servers comprising a therapy generator, the therapy generator generating a therapy session personalized to the user, the therapy session comprising an audio component;
input data from the user supplied to the therapy generator and utilized by the therapy generator to personalize the therapy session;
a user computing device receives the therapy session from the therapy generator and delivers the audio component of the therapy session to the user;
wherein the audio component is an audio voice signal that is delivered to the user nearly contemporaneously with the generation of the therapy session by the therapy generator; and
further wherein the audio component of the therapy session is as indistinguishable as possible from a human delivered therapy session.

9. The computing system of claim 8, wherein the input data from the user comprising one or more of the circumstances of the user selected from the group consisting of personality traits, strengths, relationships, support network, life events, varied preferences, physical location and geography.

10. The computing system of claim 8, further comprising:
a. The therapy session also including a video component synched with the audio component, wherein the combination of audio and video is as indistinguishable as possible from a video of a human speaking.

11. The computing system of claim 8, wherein the audio component is generated from voice recordings.

12. The computing system of claim 11, wherein generation of the audio component involves the therapy generator generating a therapy text that is passed through an algorithm to convert the text to the audio voice signal utilizing the voice recordings.

13. The computing system of claim 8, wherein the input data from the user is supplied to the therapy generator during the therapy session.

14. The computing system of claim 8, wherein the therapy session is selected from the group consisting of meditation therapy, behavioral therapy, yoga therapy, coaching therapy, cognitive behavioral therapy, acceptance/commitment therapy, solution focused therapy, behavior activation therapy, mindfulness based stress reduction therapy and behavior therapy.

15. A customizable therapy system, comprising:
a therapy generator automatically generating a therapy session for delivery to the user, the therapy session comprising an audio component and being personalized to a user;
a user computing device selected from the group consisting of a smartphone, tablet, personal computer and smart device, the user computing device receiving input data from the user, the input data supplied to the therapy generator and utilized by the therapy generator to personalize the therapy session;
the user computing device receives the therapy session from the therapy generator and delivers the audio component of the therapy session to the user;
wherein the audio component is an audio voice signal that is delivered to the user nearly contemporaneously with the generation of the therapy session by the therapy generator; and
further wherein the audio component of the therapy session is as indistinguishable as possible from a human delivered therapy session.

16. The customizable therapy system of claim 15, wherein the input data from the user comprising one or more of the circumstances of the user selected from the group consisting of personality traits, strengths, relationships, support network, life events, varied preferences, physical location and geography.

17. The customizable therapy system of claim 15, further comprising:
a. The therapy session also including a video component synched with the audio component, wherein the combination of audio and video is as indistinguishable as possible from a video of a human speaking.

18. The customizable therapy system of claim 15, wherein generation of the audio component involves the therapy generator generating a therapy text that is passed through an algorithm to convert the text to the audio voice signal utilizing the voice recordings.

19. The customizable therapy system of claim 15, wherein the input data from the user is supplied to the therapy generator during the therapy session.

20. The customizable therapy system of claim 15, wherein the therapy session is selected from the group consisting of meditation therapy, behavioral therapy, yoga therapy, coaching therapy, cognitive behavioral therapy, acceptance/commitment therapy, solution focused therapy, behavior activation therapy, mindfulness based stress reduction therapy and behavior therapy.
